SEQUENCE LISTING (23 sequences)

SEQ ID NO: 1 - DNA, 4368 bp, homo sapiens
  CDS (216)..(2183)
atcattgtaaacaggcagaggctgggcggggtgggaatggggcgcccgaggccggcctggggcgcagcgcaggaggcggctccggtggctgcggcggcagcgtgagcgcgaggaggcggaggctgcggcggggcggacggtcgcgcggcggcaggcacaggtgtaatggataggtaacagagaagacctcgtcccttcctagtcagggcatcagcatgactgagtgcttcctgccccccaccagcagccccagtgaacaccgcagggtggagcatggcagcgggcttacccggacccccagctctgaagagatcagccctactaagtttcctggattgtaccgcactggcgagccctcacctccccatgacatcctccatgagcctcctgatgtagtgtctgatgatgagaaagatcatgggaagaaaaaagggaaatttaagaaaaaggaaaagaggactgaaggctatgcagcctttcaggaagatagctctggagatgaggcagaaagtccttctaaaatgaagaggtccaagggaatccatgttttcaagaagcccagcttttctaaaaagaaggaaaaggattttaaaataaaagagaaacccaaagaagaaaagcataaagaagaaaagcacaaagaagaaaaacataaagagaagaagtcaaaagacttgacagcagctgatgttgttaaacagtggaaggaaaagaagaaaaagaaaaagccaattcaggagccagaggtgcctcagattgatgttccaaatctcaaacccatttttggaattcctttggctgatgcagtagagaggaccatgatgtatgatggcattcggctgccagccgttttccgtgaatgtatagattacgtagagaagtatggcatgaagtgtgaaggcatctacagagtatcaggaattaaatcaaaggtggatgagctaaaagcagcctatgaccgggaggagtctacaaacttggaagactatgagcctaacactgtagccagtttgctgaagcagtatttgcgagaccttccagagaatttgcttaccaaagagcttatgcccagatttgaagaggcttgtgggaggaccacggagactgagaaagtgcaggaattccagcgtttactcaaagaactgccagaatgtaactatcttctgatttcttggctcattgtgcacatggaccatgtcattgcaaaggaactggaaacaaaaatgaatatacagaacatttctatagtgctcagcccaactgtgcagatcagcaatcgagtcctgtatgtgtttttcacacatgtgcaagaactctttggaaatgtggtactaaagcaagtgatgaaacctctgcgatggtctaacatggccacgatgcccacgctgccagagacccaggcgggcatcaaggaggagatcaggagacaggagtttcttttgaattgtttacatcgagatctgcagggtgggataaaggatttgtctaaagaagaaagattatgggaagtacaaagaattttgacagccctcaaaagaaaactgagagaagctaaaagacaggagtgtgaaaccaagattgcacaagagatagccagtctttcaaaagaggatgtttccaaagaagagatgaatgaaaatgaagaagttataaatattctccttgctcaggagaatgagatcctgactgaacaggaggagctcctggccatggagcagtttctgcgccggcagattgcctcagaaaaagaagagattgaacgcctcagagctgagattgctgaaattcagagtcgccagcagcacggccgaagtgagactgaggagtactcctccgagagcgagagcgagagtgaggatgaggaggagctgcagatcattctggaagacttacagagacagaacgaagagctggaaataaagaacaatcatttgaatcaagcaattcatgaggagcgcgaggccatcatcgagctgcgcgtgcagctgcggctgctccagatgcagcgagccaaggccgagcagcaggcgcaggaggacgaggagcctgagtggcgcgggggtgccgtccagccgcccagagacggcgtccttgagccaaaagcagctaaagagcagccaaaggcaggcaaggagccggcaaagccatcgcccagcagggataggaaggagacgtccatctgagcagcctgcgtggccgtctggagtccgtgagactgaaaggacccgtgcatcttactgtaacccgggggccaggccggctctctcgctgtacattctgtaaaggtgtcttctcttctcagactcttcctctgtcacacgtctgactccttcacgtcaggctcaggttccatgggaggacgaagcagtggacgcattgtgggctttagggacagatgagttttccagatagtgtcagcttatttgaagattaattttctttgttaacttaaaataactattttaacccttgagtggcttctttttaaaccaaaaaccgtctttctttgcttttttatcacagcagaatcaggatctctttctcattcaaggggggaaccaccccaggtcagcgctgcgcctgctgtggccgccgcgagccacgccctctgggatctctggtaccgtcactcttgcttgtgccttccacaccttctcggtgcagatccctatgggggagctgcctcacgttctctgactggtcagagcagcgcctggtgggtgttccctggcccactctcctctctccttctgcagttctaaaccacagtctataagcccgagtcaccaggacggcctgtctggccacagacaggggctgcctgtggagcctgcccaccggcccccggcagtgcagtccagcggggaggaggctgcccgttcctgccagttcctcactgcggggaccagcaaaggccttctcactgggttggtcaaaggtagtcaccttggcctggtgcatccacagaggatgttgttcaaaccagaaatcttttaaacgactgaccttccttaaaaacagaatgactccgattgcttgcttgggctagaatgtacacgtctccttgcctgaataagccatatatatgctcttaaacaaaagtttgaaattatccatatcatctcagtgaacctactggtggactcccaattgacaagattgagcaatagaaaaaaattcctttcctttgaatgatagctgtgattcaccccaccccattttcttgtttctggtccatccgatgagacggatgctctgatgctctgaggcttctgggaggctgggccctggaggcaacgtgctgcaggcgcactctgtcagagtgaacagcaccgcgagacaggccaggctcgtggctcggaagacaaaccccacacacactcaaggggtcgaaaacaaaccccacacgagggctctcacctccttctcctaggtagtatttattttcagcacctgtttgatgcagtttttaatcctctacctattgcactgttgtgactcgttggccattatttgatttttgtacgaaaaaaagctttgttatagaaatcagcatactatttttttaaatctggagagaagatattctggtgactgaaagtatggtcgggtgtcagatataaatgtgcaaatgccttcttgctgtcctgtcggtctcagtacgttcactttatagctgctggcaatatcgaaggttccttttttgtttgtgtaaactctaatttctatcaaggtgtcatggatttttaaaattagtatttcattacaaatgtctcagcattggttaactaatttttgccaggaccattattgatcaagcaaataaattcaacagccatttgggaaaaagaaaagcttctagtttttttgtacacattctttctgtgaggagattgagtactctgcagctggcgaggagttggttgaggcacttcttcaaggccaagggggaacacagtgttttgtttccagctcactttgtacccctcacctctgcagacacggggagaaccccggacccctggcatgcatgctggcggcggcatgcctcccttccacaagcccatgctgctgcagagggagcctgtgtttgcaaaacccagtggactgggctgggtctgctgtctgagcagctcctggctccggtgggaactgcacacaagtccactggcctggcttggccccaggcattgcaattgacagacatttgcatttcatacggtaaatgaggactcagcacagccaaccataatcagcatgtctgggatagactggtctagaataaaaatgaagtttccattgctttgtttgctttaaaaattccacaattaaaatatctgtcattgaaagcttaaaaaaaaaaaaaaa

SEQ ID NO: 2 - protein, 655 aa, homo sapiens
MTECFLPPTSSPSEHRRVEHGSGLTRTPSSEEISPTKFPGLYRTGEPSPPHDILHEPPDVVSDDEKDHGKKKGKFKKKEKRTEGYAAFQEDSSGDEAESPSKMKRSKGIHVFKKPSFSKKKEKDFKIKEKPKEEKHKEEKHKEEKHKEKKSKDLTAADVVKQWKEKKKKKKPIQEPEVPQIDVPNLKPIFGIPLADAVERTMMYDGIRLPAVFRECIDYVEKYGMKCEGIYRVSGIKSKVDELKAAYDREESTNLEDYEPNTVASLLKQYLRDLPENLLTKELMPRFEEACGRTTETEKVQEFQRLLKELPECNYLLISWLIVHMDHVIAKELETKMNIQNISIVLSPTVQISNRVLYVFFTHVQELFGNVVLKQVMKPLRWSNMATMPTLPETQAGIKEEIRRQEFLLNCLHRDLQGGIKDLSKEERLWEVQRILTALKRKLREAKRQECETKIAQEIASLSKEDVSKEEMNENEEVINILLAQENEILTEQEELLAMEQFLRRQIASEKEEIERLRAEIAEIQSRQQHGRSETEEYSSESESESEDEEELQIILEDLQRQNEELEIKNNHLNQAIHEEREAIIELRVQLRLLQMQRAKAEQQAQEDEEPEWRGGAVQPPRDGVLEPKAAKEQPKAGKEPAKPSPSRDRKETSI

SEQ ID NO: 3 - protein, 66 aa, artificial sequence
  epitope region comprising residues 154-219
LTAADVVKQWKEKKKKKKPIQEPEVPQIDVPNLKPIFGIPLADAVERTMMYDGIRLPAVFRECIDY

SEQ ID NO: 4 - protein, 15 aa, artificial sequence
  epitope region comprising residues 171-185
KPIQEPEVPQIDVPN

SEQ ID NO: 5 - DNA, 26 bp, artificial sequence
  upstream primer sequence for clone of full length RLIP
ggcggatccatgactgagtgcttcct

SEQ ID NO: 6 - DNA, 34 bp, artificial sequence
  downstream primer sequence for clone of full length RLIP
ccgctcgagtagatggacgtctccttcctatccc

SEQ ID NO: 7 - DNA, 30 bp, artificial sequence
  mutagenic upstream primer for deletion mutant 203-219
gtagagaggaccatggtagagaagtatggc

SEQ ID NO: 8 - DNA, 31 bp, artificial sequence
  mutagenic upstream primer for deletion mutant 154-171
gaagaagtcaaaagacaagccaattcaggag

SEQ ID NO: 9 - DNA, 28 bp, artificial sequence
  mutagenic upstream primer for deletion mutant 171-185
gaagaaaaagaaactcaaacccattttt

SEQ ID NO: 10 - DNA, 31 bp, artificial sequence
  mutagenic upstream prime for deletion mutant 154-219
gaagaagtcaaaagacgtagagaagtatggc

SEQ ID NO: 11 - DNA, 31 bp, artificial sequence
  mutagenic upstream primer for deletion mutant 415-448
gaattgtttacatcgacaggagtgtgaaacc

SEQ ID NO: 12 - DNA, 28 bp, artificial sequence
  mutagenic upstream primer for deletion mutant 65-80
gtgtctgatgataggactgaaggctatg

SEQ ID NO: 13 - DNA, 23 bp, artificial sequence
  targeted cDNA corresponding to nucleotides 508-528 of ral A binding protein 1
aagaaaaagccaattcaggagcc

SEQ ID NO: 14 - RNA, 23 nt, artificial sequence
  corresponding sense siRNA strand for targeted cDNA
gaaaaagccaauucaggagccdd

SEQ ID NO: 15 - RNA, 23 nt, artificial sequence
  corresponding antisense siRNA strand for targeted cDNA
ggcuccugaauuggcuuuuucdd

SEQ ID NO: 16 - RNA, 23 nt, Artificial Sequence
  scrambled sense siRNA strand
guaacugcaacgauuucgaugdd

SEQ ID NO: 17 - RNA, 23 nt, artificial sequence
  scrambled antisense siRNA strand
caucgaaaucguugcaguuacdd

SEQ ID NO: 18 - DNA, 21 bp, artificial sequence
  phosphorothioate antisense oligonucleotides
ggctcctgaattggctttttc

SEQ ID NO: 19 - DNA, 22 bp, artificial sequence
  corresponding silencing RNA sequence to the phosphorothioate antisense oligonucleotide
aagaaaagccaattcaggagcc

SEQ ID NO: 20 - DNA, 198 bp, artificial sequence
  epitope nucleotide sequence corresponding to residues 154-219
ttgacagcagctgatgttgttaaacagtggaaggaaaagaagaaaaagaaaaagccaattcaggagccagaggtgcctcagattgatgttccaaatctcaaacccatttttggaattcctttggctgatgcagtagagaggaccatgatgtatgatggcattcggctgccagccgttttccgtgaatgtatagattac

SEQ ID NO: 21 - DNA, 45 bp, artificial sequence
  epitope nucleotide sequence corresponding to residues 171-185
aagccaattcaggagccagaggtgcctcagattgatgttccaaat

SEQ ID NO: 22 - DNA, 1968 bp, homo sapiens
  CDS (1)..(1965)
atgactgagtgcttcctgccccccaccagcagccccagtgaacaccgcagggtggagcatggcagcgggcttacccggacccccagctctgaagagatcagccctactaagtttcctggattgtaccgcactggcgagccctcacctccccatgacatcctccatgagcctcctgatgtagtgtctgatgatgagaaagatcatgggaagaaaaaagggaaatttaagaaaaaggaaaagaggactgaaggctatgcagcctttcaggaagatagctctggagatgaggcagaaagtccttctaaaatgaagaggtccaagggaatccatgttttcaagaagcccagcttttctaaaaagaaggaaaaggattttaaaataaaagagaaacccaaagaagaaaagcataaagaagaaaagcacaaagaagaaaaacataaagagaagaagtcaaaagacttgacagcagctgatgttgttaaacagtggaaggaaaagaagaaaaagaaaaagccaattcaggagccagaggtgcctcagattgatgttccaaatctcaaacccatttttggaattcctttggctgatgcagtagagaggaccatgatgtatgatggcattcggctgccagccgttttccgtgaatgtatagattacgtagagaagtatggcatgaagtgtgaaggcatctacagagtatcaggaattaaatcaaaggtggatgagctaaaagcagcctatgaccgggaggagtctacaaacttggaagactatgagcctaacactgtagccagtttgctgaagcagtatttgcgagaccttccagagaatttgcttaccaaagagcttatgcccagatttgaagaggcttgtgggaggaccacggagactgagaaagtgcaggaattccagcgtttactcaaagaactgccagaatgtaactatcttctgatttcttggctcattgtgcacatggaccatgtcattgcaaaggaactggaaacaaaaatgaatatacagaacatttctatagtgctcagcccaactgtgcagatcagcaatcgagtcctgtatgtgtttttcacacatgtgcaagaactctttggaaatgtggtactaaagcaagtgatgaaacctctgcgatggtctaacatggccacgatgcccacgctgccagagacccaggcgggcatcaaggaggagatcaggagacaggagtttcttttgaattgtttacatcgagatctgcagggtgggataaaggatttgtctaaagaagaaagattatgggaagtacaaagaattttgacagccctcaaaagaaaactgagagaagctaaaagacaggagtgtgaaaccaagattgcacaagagatagccagtctttcaaaagaggatgtttccaaagaagagatgaatgaaaatgaagaagttataaatattctccttgctcaggagaatgagatcctgactgaacaggaggagctcctggccatggagcagtttctgcgccggcagattgcctcagaaaaagaagagattgaacgcctcagagctgagattgctgaaattcagagtcgccagcagcacggccgaagtgagactgaggagtactcctccgagagcgagagcgagagtgaggatgaggaggagctgcagatcattctggaagacttacagagacagaacgaagagctggaaataaagaacaatcatttgaatcaagcaattcatgaggagcgcgaggccatcatcgagctgcgcgtgcagctgcggctgctccagatgcagcgagccaaggccgagcagcaggcgcaggaggacgaggagcctgagtggcgcgggggtgccgtccagccgcccagagacggcgtccttgagccaaaagcagctaaagagcagccaaaggcaggcaaggagccggcaaagccatcgcccagcagggataggaaggagacgtccatctga

SEQ ID NO: 23 - protein, 655 aa, homo sapiens; identical to SEQ ID NO: 2

(19) United States
(12) Patent Application Publication
(10) Pub. No.: US 2006/0104982 A1
(43) Pub. Date: May 18, 2006

(54) Therapies for seizure disorders using RLIP76

(75) Inventors: Sanjay Awasthi, Arlington, TX (US); Sharad S. Singhal, Arlington, TX (US); Sushma Yadav, Arlington, TX (US)

Correspondence Address: GARDERE WYNNE SEWELL LLP;INTELLECTUAL PROPERTY SECTION, 3000 THANKSGIVING TOWER, 1601 ELM ST, DALLAS, TX, 75201-4761, US

(73) Assignee: Board of Regents, The University of Texas System, Austin, TX (US)

(21) Appl. No.: 11/265,052

(22) Filed: Nov. 2, 2005

Related U.S. Application Data
(63) Continuation-in-part of application No. 10/714,506, filed on Nov. 13, 2003.
(63) Continuation-in-part of application No. 10/713,578, filed on Nov. 13, 2003.
(60) Provisional application No. 60/425,917, filed on Nov. 13, 2002.
(60) Provisional application No. 60/425,814, filed on Nov. 13, 2002.

(51) Int. Cl. A61K 39/395; C40B 40/08; A61K 48/00

(52) U.S. Cl. 424/155.1; 514/44; 435/6

(57) ABSTRACT

The present invention is a composition identified as a coding region of ralA binding protein 1, wherein the region neighbors a membrane-associated portion of the ralA binding protein 1 and directly effects transport activity and membrane association of the ralA binding protein 1. The composition is used to identify chemical compounds (e.g., antibodies, si-RNA and small molecules) that recognize ralA binding protein 1. The composition and methods of using the composition identify, via screening of chemical libraries, compounds that bind ralA binding protein 1 and medicines for the treatment of seizure disorders.

### CROSS-REFERENCES TO RELATED APPLICATIONS

This application is a continuation-in-part of prior U.S. application Ser. No. 10/714,506, filed Nov. 13, 2003, herein incorporated by reference, which claims the benefit of U.S. Provisional Patent Application No. 60/425,917, filed Nov. 13, 2002. This application is a continuation-in-part of prior U.S. application Ser. No. 10/713,578, filed Nov. 13, 2003, herein incorporated by reference, which claims the benefit of U.S. Provisional Patent Application No. 60/425,814, filed Nov. 13, 2002.

### STATEMENT REGARDING FEDERALLY SPONSORED APPLICATIONS

The U.S. Government has a paid-up license in this invention and the right in limited circumstances to require the patent owner to license others on reasonable terms as provided for by the terms of Grant No. NIH 2-R01-CA77495, Grant No. NIH CA 104661.

### REFERENCE TO A SEQUENCE LISTING

This application incorporates by reference sequence listing material included on computer readable form and identified as 124263-1038 RLIP.ST25.txt saved on November 1, in ASCII readable form.

### BACKGROUND OF THE INVENTION

The present invention relates to improved therapies for seizure disorders and related neurologic disorders, particularly those stemming from the brain.

Crossing the blood brain barrier in order to offer therapeutic assistance to areas of an affected brain have proven difficult, particularly in persons exhibiting multiple drug resistance. This is because a therapeutic agent must pass through specialized cells lining the blood-brain barrier known as vascular endothelial cells in order to enter the brain where it can exert its effects. Unfortunately, these endothelial cells typically pump such agents back into the blood vessel lumen, thereby preventing entry of the agent into the brain and promoting resistance to that agent. Because of the difficulties imposed by the blood-brain barrier, many current therapies aimed at improving symptoms of seizures and other neurologic disorders are either surgical or those that modify behavior, diet or stimulate the vagal nerve. Medicinal agents, such as ion channel agonists and potentiators of gamma aminobutyric acid remain under investigation. For those approved by the Food and Drug Administration, data provided by the National Institutes of Health indicate that more than 20% of persons diagnosed with a seizure disorder have seizures that cannot be controlled by surgical or medicinal therapies. Moreover, each year, at least I of sixteen persons diagnosed with a seizure disorder develops treatment-resistance according to statistics provided by the Columbia University and the Institute of Neurology, University College London. Therefore, there remains a need for improved therapies for seizure disorders and related neurologic disorders, particularly those capable of crossing the blood-brain barrier and that do not promote resistance to treatment.

### SUMMARY OF THE INVENTION

The present invention solves problems associated with entry of medicinal agents into the central nervous system. As provided herein, RLIP76 is found to be membrane associated protein at the blood-brain barrier and a critical as well as predominant regulator of anti-seizure medicines and their entry into the central nervous system. Accordingly, chemicals and molecules that directly effect RLIP76 activity and/or its association with the membrane are effective medicines for seizure-related disorders.

In one form, the present invention provides for a critical region of ralA binding protein 1, wherein the region neighbors a membrane-associated portion of the ralA binding protein 1 and directly affects transport activity and membrane association of the ralA binding protein 1. The region is used for screening of chemical compounds to be used as medicinal agents for the prevention and treatment of seizure-related disorders.

Compositions of the present invention include an internal peptide region of RLIP76 to be used as bait in screens of chemical libraries for synthetic and naturally occurring organic chemicals and compounds with anti-seizure activity. The identified chemicals and compounds are those acting as specific inhibitors of RLIP76 activity (e.g., found to inhibit RLIP transport of glutathione). As such, the present invention provides for compositions that are improved therapies for seizure-related disorders.

In one form, there present invention provides for a coding region of ralA binding protein 1, wherein the region further comprises all or a portion of SEQ ID NO.:20.

In other forms, the present invention provides for compositions including SEQ ID NO.: 3 to SEQ ID NO.:19 and SEQ ID NO.:21. Such compositions, in various forms may be used to identify compounds as anti-seizure medicines.

Those skilled in the art will further appreciate the above-noted features and advantages of the invention together with other important aspects thereof upon reading the detailed description that follows in conjunction with the drawings.

### BRIEF DESCRIPTION OF THE DRAWINGS

For more complete understanding of the features and advantages of the present invention, reference is now made to the detailed description of the invention along with the accompanying figures, wherein:

FIG. 1 depicts representative immunoreactivity of RLIP with blood vessels and blood cells obtained from normal human donors and donors with a seizure disorder;

FIG. 2 depicts a representative example of liposomal uptake of phentytoin (PHE) in the presence and absence of RLIP76 and adenosine triphosphate (ATP);

FIG. 3 depicts a representative example of time-dependent uptake of ¹⁴C-PHE and ¹⁴C-CBZ by RLIP76-proteoliposomes in the presence of ATP;

FIG. 4 depicts saturable kinetics of ¹⁴C-PHE and ¹⁴C-CBZ transport by purified recombinant human RLIP76 with respect to various ATP concentrations;

FIG. 5 depicts a representative example of transport of PHE and CBZ by proteoliposomes comprising RLIP76;

FIG. 6 depicts Kₘ values for RLIP76-mediated transport of ¹⁴C-PHE and ¹⁴C-CBZ;

FIG. 7 depicts a representative example of mRNA levels of RLIP76 in normal donor endothelial cells and those from donors with a seizure disorder;

FIG. 8 depicts (A) RLIP76 protein levels and its correlation with (B) transport activity of PHE in inside-out membrane vesicles prepared from normal and seizure disorder donor brain tissue;

FIG. 9 and FIG. 10 depict representative examples of the relative contribution of RLIP76 and MDR1 to transport of PHE or CBZ in inside-out vesicles prepared from brain tissue of non-seizure, non-resistant donors and seizure, multiple resistant donors;

FIG. 11 and FIG. 12 depict time-dependent PHE uptake by inside-out vesicles prepared from primary cultures of astrocytes or endothelial cells from non-seizure donor brains (diamond) or seizure donor brains (square);

FIG. 13 and FIG. 14 depict brain levels of PHE in RLIP76^{+/+} and in RLIP76^{±} mice;

FIG. 15 depicts a schematic of RLIP activity at the blood brain barrier; and

FIG. 16 depicts a schematic of one manner in which the present invention provides for RLIP-specific inhibitors as improved therapies for seizure disorders.

### DETAILED DESCRIPTION OF THE INVENTION

Although making and using various embodiments of the present invention are discussed in detail below, it should be appreciated that the present invention provides many inventive concepts that may be embodied in a wide variety of contexts. The specific aspects and embodiments discussed herein are merely illustrative of ways to make and use the invention, and do not limit the scope of the invention.

In the description which follows like parts are marked throughout the specification and drawing with the same reference numerals, respectively. The drawing figures are not necessarily to scale and certain features may be shown exaggerated in scale or in a somewhat generalized or schematic form in the interest of clarity and conciseness.

The following are abbreviations that may be used in describing the present invention: RLIP, ral interacting protein; MDR, multi-drug resistance; GS-E, glutathione-electrophile conjugates; phenytoin, PHE; carbamazepine, CBZ.

The causes of seizure-related disorders are numerous. When found to be resistant to treatment, the resistance may be related to a number of mechanisms, such as ontogenic abnormalies in brain maturation, severe brain injuries with resultant irreversible changes to cerebral neuroglial organization and inhibitory neuron function, kindling phenomenon, seizure-induced disturbances of oxygen supply, as well as acquired (or hereditary) changes in transporter proteins of the blood-brain barrier which function in the efflux of anti-seizure medicines from the brain. The latter mechanisms have been the focus of intense efforts to develop new rationally designed therapies that bypass such transport mechanisms. To this end, the ABC-family of transporters have become a subject of considerable interest in understanding mechanisms of treatment resistance in seizure disorders. A prototypical ABC transporter, P-glycoprotein (also known as Pgp, MDR1 and mdr-1 gene product), is highly expressed in the blood-brain barrier and in the endothelial layer, as are other transporters, including multidrug resistance-associated protein-2 (MRP-2) and breast cancer resistance protein (BCRP). Unfortunately, the relative contributions of the different ABC transporters in mediating resistance to treatment remains unknown and has become a major impediment in the development of improved and targeted therapeutic agents.

The inventors, Sanjay Awasthi and Sharad S. Singhal, of the present invention have recently described a novel non-ABC transporter that appears to be multispecific as a Ral interacting protein (see U.S. application Ser. No. 10/714,506; U.S. application Ser. No. 10/713,578; each incorporated herein by reference). As used herein, this transporter is referred to RLIP76 or RalBP1. The official human genome name for the protein is RALBP1 (SEQ ID NO.:1; and SEQ ID NO.:22 for the coding sequence). RLIP76 is a modular multifunctional and modular protein found ubiquitously in many species from Drosophila to humans. It is encoded in humans on chromosome 18p11.3 by a gene with 11 exons and 9 introns. The protein product, also known as ralA binding protein 1, is typically a 76 kDa (SEQ ID NO.:2; and SEQ ID NO.:23 for the coding sequence) protein; however, splice-variants including a 67 kDa peptide and a longer 80 kDa or 102 kDa peptide, cytocentrin, have also been identified. RLIP76 has been identified as providing an efflux mechanism for removing glutathione-electrophile conjugates (GS-E, such as LTC4) from cells as described by the inventors in Awasthi et al. Biochemistry 2000;39:9327-9334. The present invention provides a further role of RLIP76 in mediating the transport of anti-seizure medicines along the blood-brain barrier.

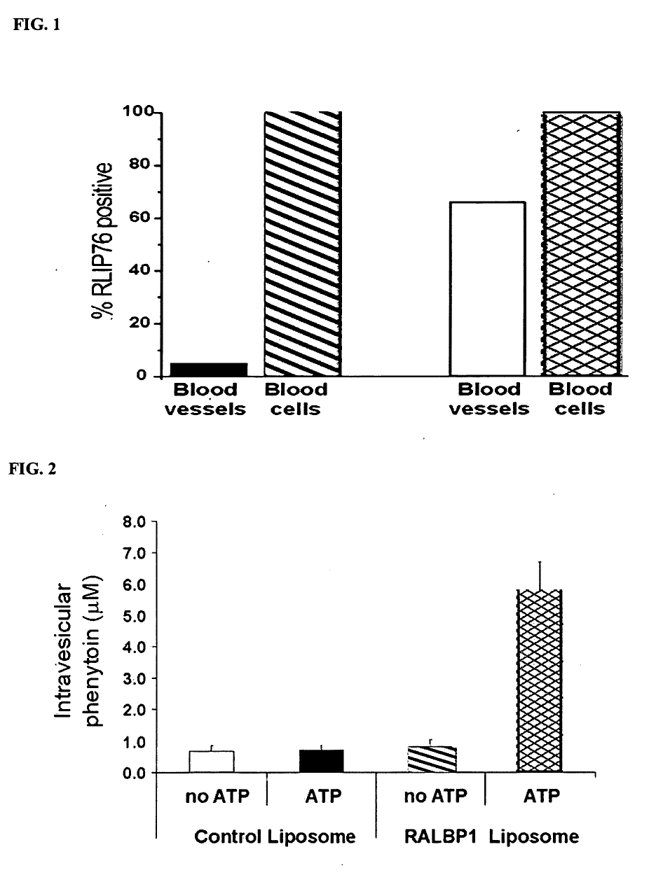

Immunohistochemical analysis of tissue sections obtained from normal autopsied subjects showed that RLIP76 was virtually absent from normal brain autopsies (both gray and white matter were analyzed). RLIP76 was barely detectable in normal brain parenchyma or vessels. On the other hand, blood vessels from epileptic patients with multiple drug resistance had a markedly increased expression of RLIP76, as shown in FIG. 1, in which both vascular and intravascular cells were RLIP76-immunopositive. RLIP76-positive intravascular cells were anucleate and did not react with the nuclear stain DAPI (data not shown). In addition, RLIP76 was expressed exclusively in epileptic endothelial cells, limited to cortical vessels, and did not localize to glia or neurons. Cumulative data for brain and blood tissue are presented in FIG. 1 in which the black bar is control blood vessels, cross-hatched bar is control blood cells, white bar is epileptic blood vessels and x-hatched bar is epileptic blood cells.

Epileptic brain sections obtained from treatment resistant patients also revealed substantial differences compared to non-epileptic brain. By immuno-cytochemical analysis, there was widespread RLIP76 expression in the cerebral vasculature (endothelium) from epileptic brain. Double label immuno-fluorescence revealed RLIP76 co-localized with the MDR1 (particularly at the luminal surface of the endothelium) but not with the neuronal nuclear antigen (NeuN) or glial fibrillary acidic protein (GFAP) (data not shown). Yet, while MDR1 was also expressed in both parenchymal glia as well as endothelial cells, RLIP76 immuno-reactivity was limited to the vasculature (e.g., large pial vessels and capillary-size vessels). No overlapping expression of RLIP76 was observed in GFAP-positive astroglia or NeuN-positive neurons (data not shown). By confocal analysis, RLIP76 expression was found in capillary endothelial cells, penetrating pial vessels, and larger (>100 μm) vessels (data not shown). In capillary endothelial cells, MDR1 expression was both lumenal (endothelial) and abluminal (glial endfeet), whereas RLIP76 expression was predominantly lumenal and did not co-localize with GFAP immunoreactivity (data not shown). Tissue from over 40 human donor subjects with epilepsy were used, including samples from 6 autopsies (see the Table). The age, sex and drug resistance was available for all subjects except those obtained from autopsy material. Non-epileptic subjects were either undergoing surgery for aneurysm clipping or to remove arteriovenous or other vascular malformations. None had experienced seizures prior to surgery and none had received antiepileptic drug treatment. Drug resistance data for subjects having received both medicinal and surgical therapy was: CBZ=61%; PHE=77%; pentobarbital (PBT)=55%; topiramate (TPM)=50%; valproic acid (VAL)=44%; other ASDs, less than 5%. Most patients were resistant to surgical and medicinal treatment; the majority having resistance to multiple drugs.

TABLE
Tissue Donor Information

| ID | Age | Sex | Disorder | Treatment failure | Use |
|---|---|---|---|---|---|
| 1 | 0.8 | M | Epi-Hemispherectomy | None | WB |
| 2 | 0.3 | M | Epi-Hemispherectomy | Ketogenic Diet only | WB; transport |
| 3 | 57 | F | Temporal Lobe Epilepsy | PHE, CBZ, TPM | WB |
| 4 | 33 | F | Control (AVM) | None | WB |
| 5 | 61 | F | Temporal Lobe epilepsy | VAL, CBZ, TPB | WB; transport |
| 6 | 30 | M | Control Parasaggital Cyst | None | WB; transport |
| 7 | 0.9 | F | Frontal/Parietal Epilepsy | VAL, PBT, TPM | WB |
| 8 | 14 | M | Temporal Lobe Epilepsy | CBZ, LEV, PBT, TPM, VAL | WB |
| 9 | 42 | M | Temporal Lobe Epilepsy | VAL, PBT, PRM, TIA | transport |
| 10 | 27 | F | Temporal Lobe Epilepsy | PHE, VAL, GBP, CBZ, TPM | transport |
| 11 | 7 | M | R. Lateral Parietal/Occipital/Temporal Lobes | PBT, PHE, TPM, CBZ, DZP, LMT | transport |
| 12 | 14 | M | Temporal Lobe Epilepsy | VAL, LEV, CBZ, other | transport |
| 13 | 31 | F | Temporal Lobe Epilepsy | GBP, CBZ, VAL, PHE, PBT | transport |
| 14 | 60 | F | Temporal Lobe Epilepsy | PBT, MBL, CBZ, DPT, VLM, MSL, FBT | In vitro |
| 15 | 43 | F | Focal Epi | N/A | In vitro |
| 16 | 56 | M | Focal Epi | PHE, PBT, CBZ, VAL, ETS | In vitro |
| 17 | 43 | F | Temporal Lobe Epilepsy | PHE, CBZ, VAL, GBP | mRNA analysis |
| 18 | 40 | F | Control (Aneurysm) | NONE | mRNA analysis |
| 20 | 58 | F | Focal Epi | PHE, PBT, VAL, PRM, FBT, GBP, TPM | mRNA analysis |
| 21 | 35 | M | Temporal Lobe Epilepsy | PHE, CBZ, VAL, PRM, VGB | mRNA analysis |
| 22 | 28 | M | Temporal Lobe Epilepsy | PHE, PBT, CBZ, VAL, TPM, CZP, GBP | mRNA analysis |
| 23 | 38 | M | Temporal Lobe Epilepsy | PHE, CBZ, VAL, FBM, GBP | mRNA analysis |
| 24 | 39 | M | Temporal Lobe Epilepsy | PHE, PBT, VAL, PRM, MSD | mRNA analysis |
| 25 | 31 | M | Temporal Lobe Epilepsy | PHE, PBT, VAL, TPM | mRNA analysis |
| 26 | 23 | M | Temporal Lobe Epilepsy | PHE, PBT, CBZ, VAL, GBP | mRNA analysis |
| 27 | 45 | F | Control (Aneurysm) | NONE | mRNA analysis |
| 28 | 22 | F | Control (Aneurysm) | NONE | mRNA analysis |
| 36 | 7 | M | Temporal Lobe Epilepsy | PHE, CBZ, VAL, GBP | ICC |
| 37 | 28 | F | Temporal Lobe Epilepsy | PHE, PBT, CBZ, VAL | ICC |
| 38 | 7 | M | Temporal Lobe Epilepsy | ? | ICC |
| 39 | 4 | F | Frontal and Parietal Epilepsy | PHE, CBZ, VAL, PRM, | ICC |
| 40 | 8 | F | Temporal lobe epilepsy | CBZ, PHE, VAL | ICC |
| 41 | 24 | M | Control (AVM) | NONE | ICC |
| 42 | 1 | M | Frontal Epilepsy | CBZ, PHE, VAL | ICC |
| 43 | 25 | M | Temporal Lobe Epilepsy | PHE, CBZ | ICC |
| 44 | 1 | M | Hemispherectomy | None | ICC |
| 45 | 23 | F | Contol (AVM) | None | ICC |
| 46 | 23 | M | Temporal lobe epilepsy | GBP, CBZ, VAL, PHE | ICC |
| 47 | 23 | M | Control (AVM) | None | ICC |
| 48 | 7 | M | TSC | ? | ICC |
| 49 | 3 | F | Epilepsy Right Hemi | ? | ICC |
| 50 | | | Autopsy | | ICC |
| 51 | | | Autopsy | | ICC |
| 52 | | | Autopsy | | ICC |
| 53 | | | Autopsy | | ICC |
| 54 | | | Autopsy | | ICC; WB |
| 55 | | | Autopsy | | ICC; WB |

For the Table: AVM=arteriovenous malformations; Epi epilepsy; Hemi=hemisphere; WB=Western blot analysis; transport =drug transport study; in vitro=in vitro ASD studyl ICC=immunocytochemistry; GBP=gabapentin; FBT=felbamate; ETS=ethosuximide; DZP=diazepam; LEV=levetiracetam; LMT=lamotrigine; MSD=musculo-skeletal disorder; PRM=primidone; TSC=tuberous sclerosis complex (non-drug); TIA=tiagabine; TSC.

Donor tissue sample extraction and use conformed to the principles outlined in the Declaration of Helsinki. For freshly isolated surgical samples, patient consent was obtained as per the Institutional Review Board instructions before samples were collected. Autopsy materials were obtained from organ donors. Endothelial cells and glia were isolated from brain specimens from patients undergoing temporal lobectomies to relieve medically intractable seizures (n=12) or cortices of patients undergoing surgery for removal of vascular malformations (n=3). Blood vessels were isolated from resected tissue by manually pulling out a combination of penetrating pial and superficial pial vessels using methods know to one of ordinary skill in the art.

For immunochemistry analysis, the anti-RLIP76 antibody was a polyclonal rabbit-anti-RLIP76 IgG prepared and purified by methods know to one of ordinary skill in the art. Anti-MDR1 antibodies was a goat-anti-human Pgp antibody C-19 purchased from Santa Cruz Biotech in Californai. For RLIP76 antibodies, recombinant human RLIP76 was expressed in E. coli and purified by DNP-SG-Sepharose affinity purification. Approximately 75 μg was injected into New Zealand White rabbits after obtaining pre-immune serum. After booster doses of 50 μg each at two-week intervals, post-immune serum was obtained. The IgG fraction from pre- and post-immunized heat-inactivated serum was purified by DE-52 anion exchange chromatography, followed by protein-A-Sepharose affinity chromatography. The purity of the antibody was checked by SDS-PAGE as well as Western blotting against goat anti-rabbit IgG. Aliquots of the antibody were stored at −86° C. and checked regularly by aerobic and anaerobic cultures for contamination. The specificity of anti-RLIP and other antibodies were stringently established: purified recombinant RLIP76 used for raising polyclonal antibodies was demonstrated to be homogenous by amino acid composition analysis and showed amino acid yields within 96% of those expected according to its sequence SELDI-MS demonstrating a pattern of [M+H] peaks consistent with homogenous preparations of RLIP76 (data not shown).

Localization of RLIP76 protein in donor tissue samples were performed using methods known to one of ordinary skill in the art. In brief, slide mounted sections of frozen tissue (approximately 10 μm thick) were labeled with purified anti-RLIP76 IgG as the primary antibody and FITC-conjugated purified donkey anti-rabbit IgG (Jackson Immuno Research Laboratories, Pennsylvania) as the secondary antibody.

A system comprising artificial liposomes reconstituted in the presence of purified human RLIP76 was used to demonstrate RLIP76 activity in the presence of anti-seizure agents, such as PHE and CBZ. Uptake of PHE by liposomes with RLIP76 or without (control) was examined in the presence or absence of ATP (in the transport medium). ATP increased the uptake of ¹⁴C-PHE by RLIP76-liposomes in a dose dependent fashion, whereas ATP had no effect on uptake by control liposomes as shown in FIG. 2. The intra-vesicular concentration of PHE in control liposomes (with or without ATP) and in RLIP76 liposomes without ATP were near the extra-vesicular drug-concentration (1 μM). RLIP76-liposomes in the presence of ATP had intra-vesicular PHE concentrations of at least about 5.7 μM, demonstrating that in the presence of ATP, RLIP76 liposomes are able to concentrate PHE against a gradient, the hallmark of active transport. Interestingly, it has been commonly believed that PHE was a substrate for MDR1.

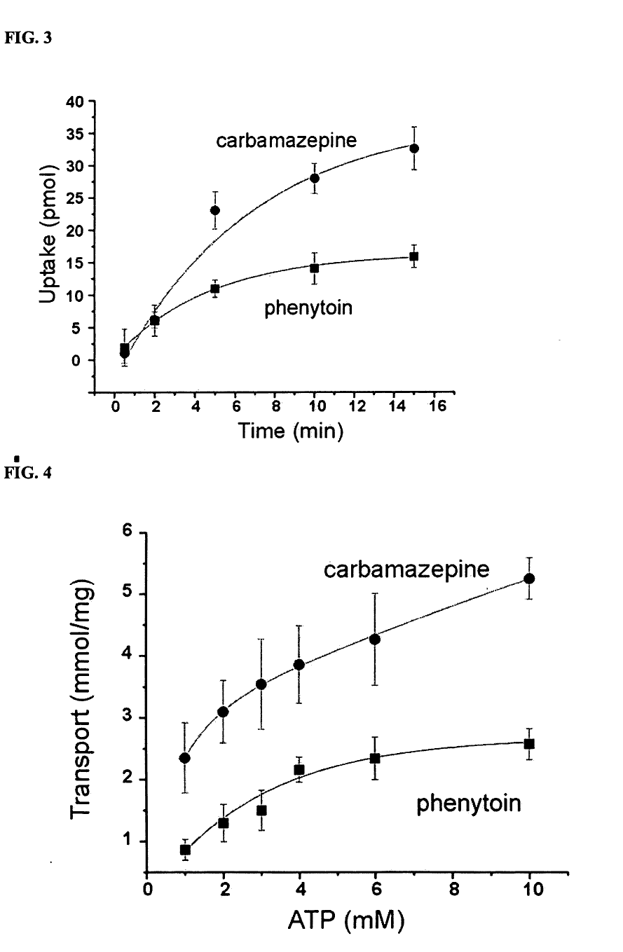

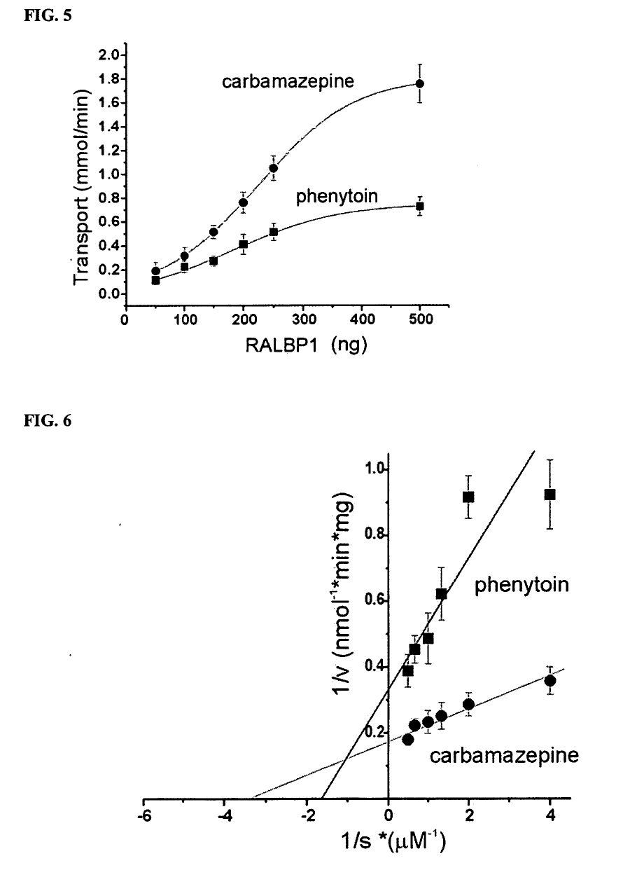

Similar findings to those observed with PHE were obtained with the transport of CBZ. Uptake of both PHE or CBZ by inside-out vesicles was a time dependent process having kinetics consistent with a single compartment filling model (see FIGS. 3 and 4), and the initial velocity of transport could be reasonably estimated by measuring uptake at two minutes after addition of ATP. Clearly, more than one anti-seizure medicine could be transported by RLIP76 as shown in FIG. 5. Initial velocity kinetics performed by varying one substrate (either ATP or CBZ or ATP or PHE) while holding the other constant showed that the Kₘ for PHE and CBZ was about 0.43 μM and 0.25 μM, respectively, (FIG. 6). The Kₘ for ATP was 1.33 mM with PHE and 3.3 mM with CBZ (FIGS. 4 and 6).

For liposomal preparations containing purified recombinant RLIP67, the 1968 bp full-length open reading frame cDNA of human RLIP76 was cloned from a μgt11 human bone marrow library by immuno-screening using anti-DNP-SG ATPase antibodies and subsequently subcloned into a prokaryotic expression vector (pET30a(+) from Novagen, Wisconsin), creating a plasmid containing RALBP1 free of extraneous sequences. This plasmid was transformed into a strain of E. coli [BL21 (DE3)] from which the protein was expressed after induction. DNP-SG affinity chromatography was used to obtain purified RALBP1. ATPase activity of the purified protein was performed to monitor purification and further analyzed using Western blot analysis and amino acid composition analysis. The purified protein was then dialyzed against a liposome reconstitution buffer (10 mM Tris-HCl, pH 7.4,2 mM MgCl₂, 1 mM EGTA, 100 mM KCl, 40 mM sucrose, 2.8 mM BME, 0.05 mM BHT, and 0.025% polidocanol). Liposomes comprising asolectin and cholesterol were prepared by aqueous emulsion of soybean asolectin (40 mg/mL) and cholesterol (10 mg/mL) in the reconstitution buffer by sonication. This emulsion was diluted 10-fold by addition of dialyzed RLIP76 in reconstitution buffer to achieve a final RLIP76 concentration of about 0.1 mg/mL. The reaction mixture was sonicated for about 15 seconds at 50 W. Vesiculation was initiated by addition of SM-2 Bio-beads (200 mg/mL) pre-equilibrated in the reconstitution buffer (without polidocanol). Vesiculation was then carried out for about four hours at 4° C., followed by removal of SM-2 Bio-beads by centrifugation. Vesicles were collected and analyzed for protein content, transport activity and microbial contamination. Control vesicles to measure non-specific transport were prepared using an equal amount of crude protein from E. coli not expressing RLIP76.

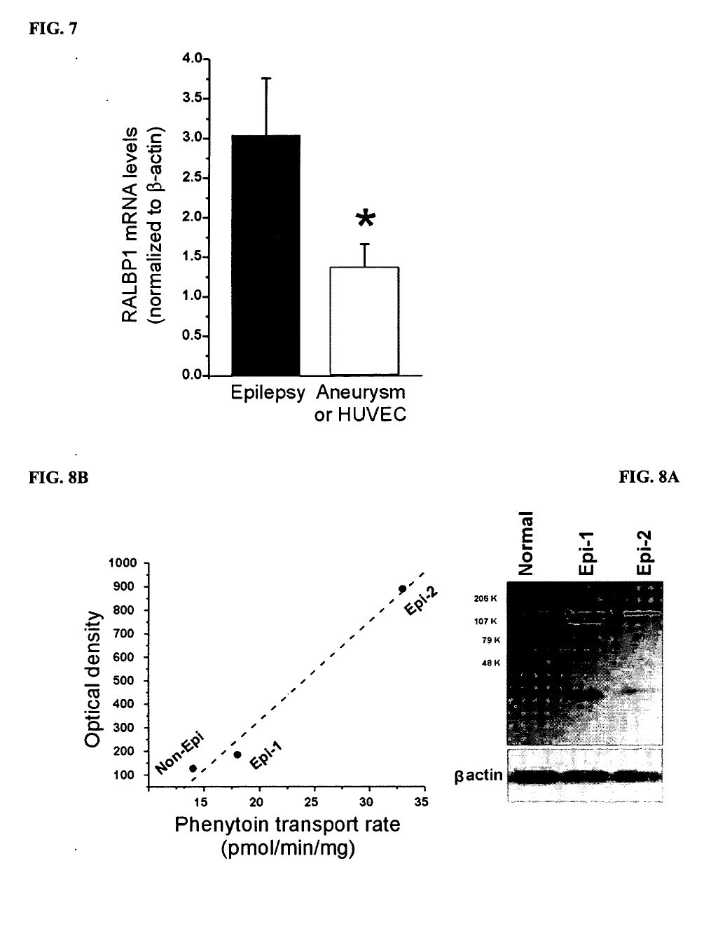

The function and level of expression of RLIP76 in non-seizure brains resected during cerebrovascular surgery (unrelated to epileptic pathology or drug resistance) as well as in seizure-disorder brains were determined. Expression was determined by Western blot of tissue blocks and mRNA analysis of isolated and cultured brain microvascular endothelial cells. RLIP76 mRNA expression was greater in brain and endothelial cells isolated from donors with a seizure disorder (FIG. 7). RLIP76 protein levels correlated with PHE transport activity measured in inside-out vesicles prepared from brain tissue as shown in FIG. 8.

For RNA extraction, surgically obtained specimens were washed in a buffered saline solution and incubated in collagenase type II (2 mg/mL at 37° C. for 20 minutes) to dissociate endothelial cells. Collagenase was washed off with a growth medium (e.g., 1.5 g/100 mL MCDB 105 from Sigma-Aldrich supplemented with Endothelial Cell Growth Supplement at 15 mg/100 mL with heparin, 800 units/100 mL, 10% fetal bovine serum, and 1% penicillin/streptomycin). Endothelial cells were then harvested using a sterile cotton swab soaked in the medium and identified as those staining positive for Von Willebrand factor and negative for glial fibrillary acidic protein. Endothelial cells were purged from the culture dishes by gentle enzymatic dissociation (collagenase) and collected by centrifugation. Total RNA was extracted with a Trizol reagent (Gibco Labs). Integrity of the isolated RNA was confirmed by agarose formaldehyde gels. For gene expression analysis, human GENEFILTERS™ (Research Genetics Inc., Huntsville, Ala.) were used, each filter containing approximately4,000 known human genes. ³³P-dCTP was used to label a probe used for hybridization procedures, as known to one of ordinary skill in the art.

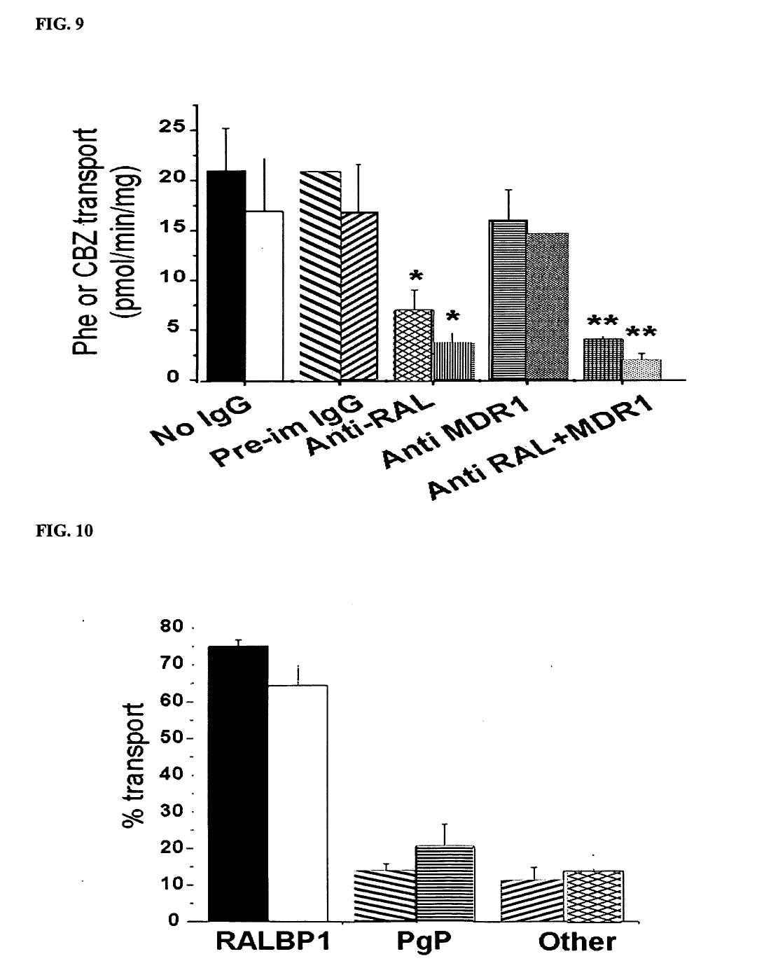

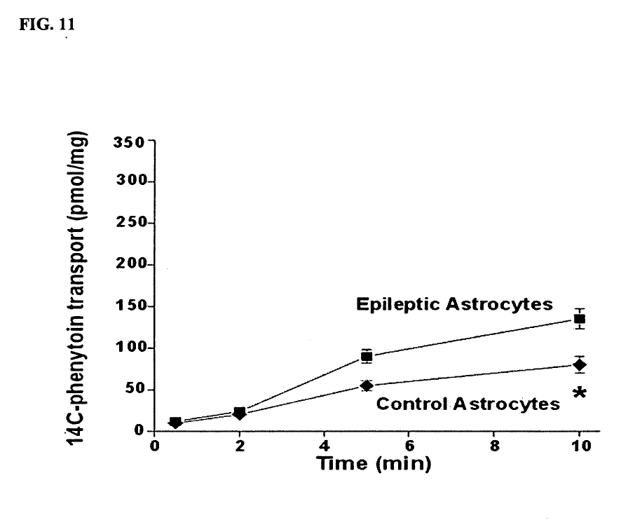

Functional expression of RLIP76 and its transport of various anti-seizure medicines were carried out using crude membrane vesicles obtained from fresh human seizure donor brain samples. Crude membrane vesicles were obtained from acutely isolated cortical samples that avoided culture-related artifacts. Transport was then determined in the absence and presence of anti-RLIP76 antibodies (FIGS. 9 and 10). For FIG. 9 and 10, the first bar in each pair shows the transport of PHE and the second bar the transport of CBZ. The transport data showed that anti-RLIP76 inhibited total transport of PHE and CBZ by about 64%±6% and 74%±1.82%, respectively, (p<0.01). When transport was repeated using anti-MDR1 antibodies, inhibition was typically 40% less for PHE (e.g., 21%±9%) and 60% less for CBZ (e.g., 13%±1.86%). Transport in the presence of both antibodies provided a cumulative inhibition. These findings indicate that RLIP76 is a predominant transporter of anti-seizure medicines in the brain.

A typical procedure for preparing crude membrane vesicles included separating human brain cells from surgically obtained specimens using a suspension buffer in which tissue specimens were placed followed by centrifugation and lysis in a hypotonic buffer (0.5 mM sodium phosphate, pH 7.0, containing 0.1 mM EDTA and 0.1 mM PMSF) for 1.5 hours followed by homogenization. The homogenate was centrifuged (12,000 g, 10 minutes at 4° C.), the postnuclear supernatant was further centrifuged (100,000 g, 40 minutes at 4° C.) and the resulting pellet was suspended in a reconstitution buffer (40 mM sucrose, 10 mM Tris-HCl, pH 7.4) followed by homogenization (layered over 38% sucrose in 5 mM Hepes-KOH, pH 7.4, using a tight fitting Dounce homogenizer). After another centrifugation (280,000 g, 2 hours at 4° C.) the interphases were collected, washed by centrifugation in the reconstitution buffer (at 100,000 g) and passed through a needle for vesicle formation.

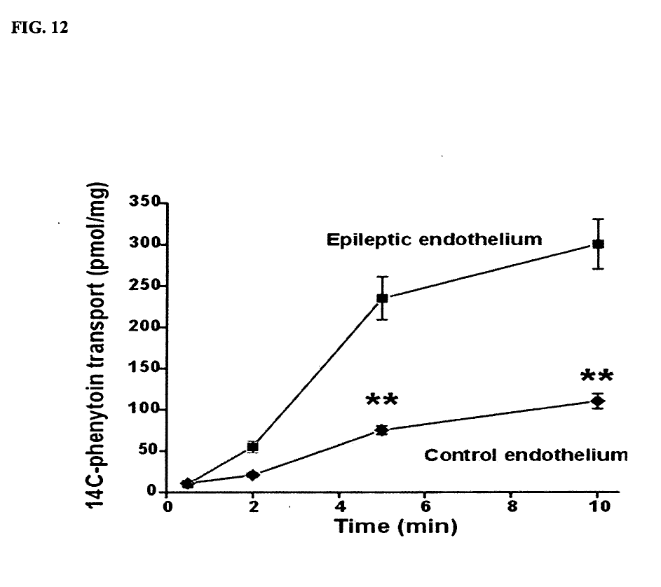

Transport of ¹⁴C-PHE in vesicles obtained from endothelial cells and from astrocytes (isolated and cultured in a similar manner) were compared using brain cells from non-seizure and seizure donors. For non-seizure donors, PHE transport was significantly higher in vesicles from endothelial cells as compared with those from astrocytes. In both types of vesicle preparations, PHE transport was even greater when obtained from seizure donors (FIGS. 11 and 12). This is in agreement with the immunocytochemical data in which the RLIP76 was found predominantly in endothelial cells and not brain parenchymal cells of seizure donors.

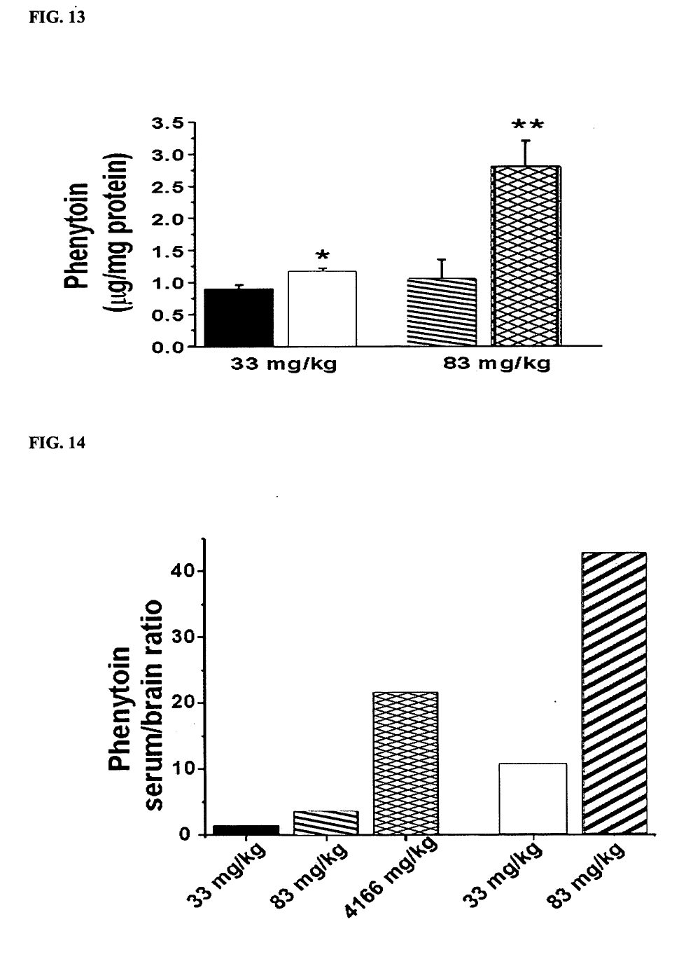

Using knock-out C57B mice lacking RLIP76 (RLIP76^{-/-}) and non-knockout C57B mice (RLIP76^{+/+} or wild-type, animals were injected intraperitoneally with PHE (33 or 83 mg/kg) and PHE accumulation in the brain and serum was evaluated (FIGS. 13 and 14). For FIG. 13, the black and cross-hatched bars are RLIP76^{+/+} and the white and x-hatched bars are RLIP76^{-/-}. For FIG. 14, the first 3 bars from the left are RLIP76+/+and the two bars on the right are RLIP76^{-/-}. Brain PHE levels were higher in RLIP76^{-/-} mice as compared with RLIP76^{+/+} (p<0.05) (FIG. 13). A statistically significant increase in PHE accumulation in brain was observed at both concentrations (FIG. 14). Overall, RLIP76^{-/-} mice were characterized by higher levels of PHE in both brain and serum compared with the wild-type, consistent with a role of RLIP76 in the renal excretion of PHE (data not shown). To account for this, a group of wild-type animals were injected with elevated doses of PHE (4166 mg/kg) to achieve serum levels comparable to those seen in the knock-out mice injected with 83 mg/kg. RLIP76^{-/-} mice demonstrated greater neurotoxicity after administration of PHE; side effects included lethargy and status-epilepticus (data not shown).

RLIP76^{±} heterozygous knockout animals were commissioned from Lexicon genetics and prepared by a strategy described previously by the inventors (Awasthi et al., Cancer Res 2005;65: 6022-6028; incorporated herein by reference). Briefly, C57B mice (about 12 weeks old and born of RLIP76^{±}×RLIP76^{±} mating) were genotyped by polymerase chain reaction strategy. C57B mice that carry heterozygous (±) or homozygous (−/−) disruption of the RLIP76 gene were generated and established colonies of RLIP76+/+, RLIP76±, and RLIP76−/− C57B mice were prepared by segregation and mating of animals based on genotyping by polymerase chain reaction on tail DNA. Western-blot analysis of various tissues using anti-RLIP76 antibodies confirmed decreased RLIP76 levels in RLIP76± mice, and its absence in tissues from RLIP76−/− mice. Consistent with an observed function of RLIP76 as a transporter of GS-E and doxorubicin (DOX) in cell culture studies, GS-E and DOX transport in membrane vesicles obtained from such mice decreased in a stepwise fashion from RLIP76+/+ mice to RLIP76± mice to RLIP76−/− mice.

For measurements of PHE concentration in serum and brain tissue obtained from wild-type and knockout mice, 12-week old C57B mice born of heterozygous with heterozygous mating were genotyped by PCR on mouse tail DNA using forward, reverse and LTR primers. PHE measurements were performed on 5 wild type (RLIP76^{+/+} ) and 5 RLIP76 knockout (RLIP76^{-/-}) animals and sacrificed 2 hours after a single intraperitoneal injection of PHE. A homogenate of mouse brain tissues was prepared and centrifuged (28,000×g, 45 minutes at 4° C.). PHE levels in homogenate and plasma was measured by applying method based on particle-enhanced turbidimetric inhibition immunoassay (PETINIA) technology and using a multichannel analyzer and a PHE Flex® reagent cartridge from Dade Behring (Illinois).

The above examples demonstrate the previously unrecognized and unanticipated activity of RLIP76 as a significant transporter of anti-seizure medicines across the blood-brain barrier. In addition, the previously unrecognized and unanticipated heightened expression and activity of RLIP76 in persons with a seizure disorder show that the transporter protein in particularly influential in person who exhibit multiple resistance to current therapies. The present invention shows that, at the blood-brain barrier, RLIP76 participates as a mediator of pharmaco-resistance in the central nervous system. This is because of the presence of RLIP76 at the anatomical interface between the brain and the vascular system; the limited yet functional expression of RLIP76 in brain endothelial cells but not parenchymal glia or neurons; the highly efficient and high level transport of anti-seizure medicines (e.g., PHE and CBZ) by RLIP76; the ability of RLIP76 to pump anti-seizure medicines out of the brain in both non-seizure donors and seizure donors; and the accumulation of an anti-seizure medicine in the central nervous system in mice lacking the RLIP76 transporter (i.e., RLIP76^{-/-} mice). The present invention demonstrates that it is not the previously expected putative mediator of multiple drug resistance, MDR1, that is active at the blood brain barrier. Instead, RLIP76, has higher potency of activity in providing resistance to therapy for persons with or without a seizure disorder. Potency of RLIP76 is enhanced with a seizure disorder.

It is speculated that RLIP76 may be linked to the pathology of seizure disorders; the protein may also act in concert or synergistically with MDR1 and MRPs (in particular MRP2). RLIP76 expression may be particularly responsive to biologic stressors and/or therapeutic insult. For example, apoptotic mechanisms are lacking in epileptic brain. Accordingly, seizure disorders may be the result of the development of foci in which cells develop one or more aberrant signaling pathways that lead to electrical (hence neurologic) instability as well as resistance to apoptosis. Certainly, increased RLIP76 confers apoptosis. In addition, increased expression of RLIP76 (particularly in those with a seizure disorder) generally corresponds with a lowering of lipid-peroxidation products, perhaps to a level that decreases the length of the absolute refractory interval between action potentials. Shortening of the refractory time (the interval during which the next full intensity signal cannot be generated) are know to cause electrical instability in other electrically active cells (i.e., cardiac conductive cells) and lead to rapid and repetitive firing of action potentials—a hallmark of seizure—or epileptic foci. In an analogous manner, RLIP76 shortens the refractory time interval for oxidative signaling (which gives rise to LTC4) by pumping such compounds out of the cell in order to return levels (e.g., of LTC4) back to a more appropriate (acceptable or pre-oxidative) level so that propagation of a subsequent signal is possible. As such, as demonstrated herein, RLIP76 partipates in regulating a signal-to-noise ratio, wherein at lower levels of noise, smaller signals can be perceived. Thus, an abnormal or stressed (e.g., oxidatively) neuron will have increased RLIP76 and be more electrically unstable. In such a model, RLIP76 is involved in a resistance paradigm and cooperates not only in the extrusion of unwanted medicines from the cell, but holds an integral role in the control of apoptosis and electrical activity of electrically active cells.

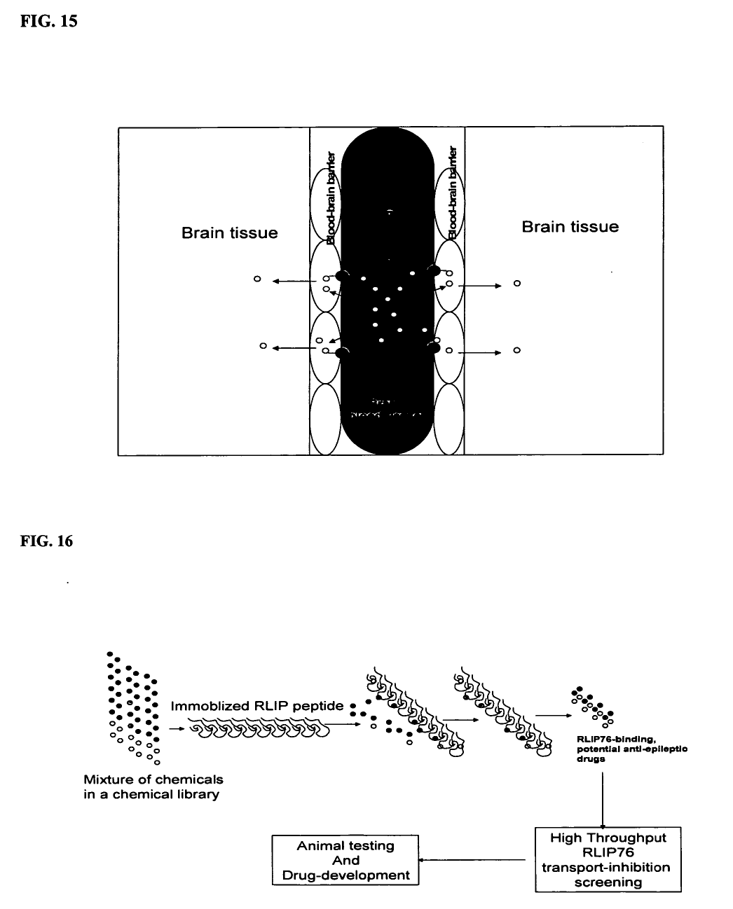

A schematic of RLIP activity at the blood brain barrier and its participation in resistance to therapy in the brain is shown in FIG. 15. In the figure, ovals represent the endothelial cells, the small circles represent molecules for transport, such as anti-seizure medicines. The figure shows that medicines, such as anti-seizure agents, that enter a vascular endothelial cell at the luminal surface of blood vessels lining the brain (i.e., blood-brain barrier) are pumped back into the blood vessel, preventing entry of the medicine into the brain tissue and, thus, initiating resistance to the agent.

Because RLIP76 appears responsible for preventing anti-seizure medicines from entering the brain, the present invention uses inhibitors of RLIP76 to be provided alone as an anti-seizure medicine or in a combination approach with another anti-seizure medicine in order to promote and enhance activity of another anti-seizure medicine. Suitable inhibitors of RLIP76 include those molecules that inhibit or reduce transport activity of RLIP76 and may be identified as described below.

The inventors have recently reported several surface epitope regions of RLIP76 when membrane bound to cells. The surface epitope region was found necessary for optimal transport activity of RLIP76 as described by Yadav et al., Biochemistry 2004;43:16243-53, herein incorporated by reference. One surface epitope region comprises on or about amino acids (aa) 154 to 219 (SEQ ID NO.:3 and modified variants, thereof, including deletions of 1 to 5 residues at the C-terminus and or N-terminus). The corresponding DNA/RNA sequence for this surface epitope region is SEQ ID NO.: 20. Another surface epitope region comprises on or about amino acids 171 to 185, corresponding to an aa sequence KPIQEPEVPQIDVPN (SEQ ID NO:4 and modified variants, thereof, including deletions of 1 to 5 residues at the C-terminus and or N-terminus, with a corresponding DNA/RNA sequence of SEQ ID NO.:21). Such surface epitope regions are not only necessary for optimal transport activity, they are also useful portions of the protein for the identification of inhibitors of RLIP76 transport activity. For example, a deletion mutant protein lacking amino acids 171 to 185 resulted in loss of hydrophobicity of the protein, decreased association of the protein with artificial liposomes, and decreased transport activity. In addition, cells transfected with 171-185 si-RNA (SEQ ID NO.:5) resulted in loss of cell surface expression (e.g., decreased membrane association).

Accordingly, the present invention identifies regions of the protein acting as surface epitopes and capable of providing inhibitors for RLIP76. Inhibitors, as identified herein, include antibodies directed against one or more surface epitope regions, si-RNA sequences directed against one or more surface epitope regions, as well as small molecules found using chemical library screenings against peptides containing one or more surface epitope regions.

In one form, surface epitope regions and their variants, as identified herein, are synthesized and immobilized on an inert support material and used to screen chemical libraries for compounds that bind this peptide as shown in FIG. 16. Suitable methods for chemical library screening are known to one of ordinary skill in the art. The compounds identified by the screening process are tested in a secondary screen that included a liposomal transport assay to determine efficiency of inhibition of RLIP76. RLIP76 inhibitors are also tested in animals alone and in combination with existing anti-seizure medicines in order to evaluate safety and efficacy of each identified inhibitor. SEQ ID NO:3 and SEQ ID NO:4 were identified from a series of deletion mutant proteins to RLIP76 (data not shown; see Yadav et al., 2004). In brief, a series of deletion mutants were prepared by PCR-based site-directed mutagenesis using a clone of the full length RLIP76 in an expression vector [pET30a(+)] as template and upstream primer 5′ GGCGGATCCATGACTGAGTGCTTCCT (SEQ ID NO.:5;:BamH1 restriction site is underlined) and downstream primer 5′CCGCTCGAGTAGATGGACGTCTCCTTCCTATCCC (SEQ ID NO.:6; XhoI restriction site underlined). Mutants included those having deletions of amino acids 203 to 219 (del 203-219), 154 to 171 (del 154-171), 171 to 185 (del 171-185), 154 to 219 (del 154-219), 415 to 448 (del 415-448) and 65 to 80 (del 65-80). The mutagenic primers for del 203-219: 5′ GTAGAGAGGACCATGGTAGAGAAGTATGGC 3′ (SEQ ID NO.: 7) with its reverse complement); for del 154-171: 5′ GAAGAAGTCAAAAGACAAGCCAATTCAGGAG (SEQ ID NO.: 8; with its reverse complement); for del 171-185: 5′ GAAGAAAAAGAAACTCAAACCCATTTTT 3′ (SEQ ID NO.: 9; with its reverse complement); for del 154-219: 5′ GAAGAAGTCAAAAGACGTAGAGAAGTATGGC 3′ (SEQ ID NO.: 10; with its reverse complement; for del 415-448: 5′ GAATTGTTTACATCGACAGGAGTGTGAAACC (SEQ ID NO.: 11; with its reverse complement); and for del 65-80: 5′ GTGTCTGATGATAGGACTGAAGGCTATG 3′ (SEQ ID NO.: 12 and its reverse complement).

The template and each deletion mutant was expressed in E. coli and after bacterial lysis (with e.g., 1% (w/v) C₁₂E₉ in lysis buffer), the protein was extracted by methods known to one of ordinary skill in the art. For the full length protein and the deletion mutants, one method of protein purification to nearly homogeneity from bacterial extracts used DNP-SG affinity resins (for full description see in Awasthi, et al. Biochemistry 2000:39:9327-9334, herein incorporated by reference). Introduction of deletions specified above in wild type RLIP76 did not effect the affinity of protein with DNP-SG; all deletion mutants could be purified by DNP-SG affinity chromatography. Protein purity was ascertained by SDS-PAGE, Western blot and amino acid composition analysis using methods know in the art and as described in Awasthi et al. 2000. The authenticity of the mutation and the absence of other fortuitous mutations were confirmed by DNA sequencing for each of the deletion mutants.

Full-length RLIP76 (wt-RLIP76) and deletion mutants (del 203-219, del 154-171, del 171-185, del 154-219, del 65-80, del 415448 and del 65-80) were expressed as recombinant (rec) proteins in E. coli (using pET30a(+) plasmid under the control of the lac UV5 promoter. Single bacterial colonies were used to induce protein expression. To facilitate extraction of the rec-RLIP76 and its various deletion mutants, bacterial lysates were collected, sonicated, and incubated. After incubation, each reaction mixture was centrifuged and the supernatant fraction was obtained as a cytosol fraction and the pellet was the membrane fraction. The membrane fraction was resuspended in 1% polidocanol (a non-ionic detergent) sonicated again, incubated and collected in the supernatant after centrifugation.

When extracted in detergent-containing buffer, the ratio of RLIP76 in the detergent/aqueous extracts was found to be 2.5 for the wild-type protein, but decreased to 0.7 in the mutant in which aa 154-219 (SEQ ID NO.:4) were deleted (data not shown; see Yadav et al., 2004). Deletion of only one segment of this region (del 171-185 or SEQ ID NO.:3) alone resulted in a significant decrease in this ratio to 1.0. For the mutants with deletions within the region from aa 154-219, loss of hydrophobicity correlated with decreased incorporation of mutants into artificial liposomes, and decreased transport activity. The data indicates that the 154-219 region of RLIP76 significantly effects protein partitioning between cytosol and membranes; Residues 171-185 contribute significantly to this effect.

Functional reconstitution of purified RLIP76 from E. coli for transport studies was performed using methods known to one of ordinary skill in the art, an example of which is described in Awasthi, et al. 2000. The degree of incorporation of wild-type as well as mutant RLIP76 into artificial liposomes was assessed by measuring RLIP76 after centrifugation (pellet and supernatant of prepared liposomes) by ELISA assay using anti-RLIP76 antibodies. Measurement of the transport of a cationic agent, doxorubicin (DOX), in the reconstituted liposomes was performed using methods known to one of ordinary skill in the art, an example of which is described in Awasthi, et al. 2000. The ATP-dependent uptake of [¹⁴C]-DOX (specific activity 8.4×10⁴ cpm/nmol) was determined by subtracting the radioactivity (cpm) of the control without ATP from that of the experimental containing ATP. Transport of DOX was calculated in terms of pmol/min/mg protein. The transport of [³H]-DNP-SG (specific activity 3.2×10³ cpm/nmol) was measured using methods known to one of ordinary skill in the art, an example of which is described in Awasthi, et al. 2000.

The majority (87%) of total wild-type RLIP76 was found in the pellet fraction, incorporated into the proteoliposomes. Deletion of aa 203-219 or 154-171 decreased incorporation slightly (to 83 and 80%, respectively). Deletion of aa 171-185 significantly effected incorporation of the protein into proteoliposomes (64%) as did deletion of residues 154-219, with only 33% of total protein found incorporated into proteoliposomes. Deletions effecting the ATP-binding sites (aa 65-80 and aa 415-448) had no significant effect on the amount of protein incorporated into proteoliposomes. Thus region 154-219 is an important determinant of membrane insertion.

For ATP-dependent transport of molecules across the proteoliposomes, transport was significantly decreased (21%) in the mutant lacking aa 154-171 (27.6 nmol/min/mg versus 21.7 nmol/min/mg for transport of DOX by the full length RLIP76 versus the deletion mutant, p<0.05). Deletion of aa 171-185 resulted in approximately 40% loss of transport activity for DOX and a similar loss (35%) in transport activity for dinitrophenyl S-glutathione (DNP-SG). Deletion of the entire 154-219 region resulted in further significant loss (50%) of transport activity for both DOX and DNP-SG. Because deletion of ATP-binding site regions did not effect partitioning of the mutants between cytosol and membrane, the observed decrease in transport activity of deletion mutant aa 154-219 is believed due to loss of protein association with the membrane because of its decreased partitioning in the membrane.

The effect in eukaryotes of losing surface epitope regions spanning residues 171-185 (SEQ ID NO.:4) or 154-219 (SEQ ID NO.:3) was similar to that described above (data not shown; see Yadav et al., 2004). When H358 cells were transfected with an empty vector (pcDNA3.1) or a vector containing either full length RLIP76 or its deletion mutants lacking aa 171-185 or aa 154-219, membrane association of RLIP76 was significantly reduced in cells transfected with the deletion mutants, as analyzed by Western blots. Hence, the aa 154-219 region is a determinant of the membrane association of RLIP76 and it is independent of whether the protein is expressed in eukaryotes or prokaryotes. Immuno-histochemistry studies using anti-RLIP76 antibodies raised against full-length RLIP76 were performed with live, unfixed H358 wild-type cells and examined by confocal laser microscopy and showed a staining pattern consistent with cell-surface localization. RLIP76 co-localized with another protein, her2/neu, known to have a cell-surface domain. Anti-RLIP76 antibody was detected using a rhodamine red-x-conjugated secondary antibody, and anti-her2/neu antibody using an FITC tagged secondary antibody. Cell-surface epitopes were recognized by both anti-RLIP76 and her2/neu antibodies which co-localized in unfixed cells indicating that RLIP76 had cell-surface epitopes just like her2/neu.

H358 cells constitutively express a wild-type RLIP76. The wild-type was removed by treating H358 cells with si-RNA directed at the region encoding aa 171-185, to silence the expression of wild-type RLIP76, while leaving the expression of 171-185 mutant unaffected. For this, a 23-nucleotide sequence motif comprising AA(N19)TT or NA(N21) (N, any nucleotide) with approximately 50% GC content was searched for. The sequence of sense si-RNA corresponds to N21. The 3′ end of the sense si-RNA was converted to TT to generate a symmetric duplex with respect to the sequence composition of sense and antisense 3′ overhangs. The selected si-RNA sequence was subjected to blast-search (NCBI database) against EST libraries, to ensure that only one gene was targeted. Chemically synthesized si-RNA duplex in the 2′ de-protected and desalted forms, was purchased from Dharmacon. A 23-nucleotide long scrambled si-RNA duplex was used as a control. The scrambled si-RNA sequence was not homologous with RLIP76 mRNA in a blast-search against RLIP76. The targeted cDNA sequence was AAGAAAAAGCCAATTCAGGAGCC (SEQ ID NO.:13) corresponding to nucleotides 508 to 528. The corresponding sense si-RNA sequence was GAAAAAGCCAAUUCAGGAGCCdTdT (SEQ ID NO.:14) and the antisense si-RNA sequence was GGCUCCUGAAUUGGCUUUUUCdTdT (SEQ ID NO.:15). The sequences of the scrambled si-RNA in the sense and antisense directions were GUAACUGCAACGAUUUCGAUGdTdT (SEQ ID NO.: 16) and CAUCGAAAUCGUUGCAGUUACdTdT (SEQ ID NO.: 17), respectively.

Transfection of si-RNA duplexes was performed using a kit (Transmessenger Transfection Reagent Kit from Qiagen) and assayed for expression about 24 hours later. Cells (approximately 3×10⁶) were placed into six-well plates and after about 24 hours were incubated for about 3 hours with RLIP76 si-RNA or scrambled si-RNA in an appropriate transfection reagent. Excess si-RNA was washed off with PBS and medium was added. Cell samples were pelleted, solubilized in a lysis buffer (10 mM Tris-HCl, pH 7.4, containing 1.4 mM P-mercaptoethanol, 100 μM EDTA, 50 μM BHT, 100 μM PMSF and 1% polidocanol), sonicated and then incubated for about 4 h in the cold (4° C.). Afterwards, each sample was centrifuged and supernatants (containing both cytosolic proteins and solubilized membrane proteins) collected and analyzed by Western blot analyses according to a method provided by Towbin et al. (Towbin, et al. PNAS 1979;76:4350-4353) using anti-RLIP76 IgG as well as IgG against the peptide 171-185. Gel bands were quantified by scanning densitometry. Polyclonal antibodies against various deleted epitope regions of RLIP76 were custom made. The peptide antibodies as well as pre-immune serum were purified by DE-52 anion exchange chromatography, followed by protein-A-Sepharose affinity chromatography to obtain pure IgG fractions. Immuno-reactivity and specificity of these peptides using their respective purified IgG were checked by dot blot analyses.

The si-RNA 171-185 effectively silenced wild-type RLIP76 expression in the untransfected, empty-vector-transfected, as well as wild-type RLIP76 transfected cells (data not shown; see Yadav et al., 2004). Antibodies against the 171-185 peptide failed to detect RLIP76 antigen, while antibodies against full-length RLIP76 recognized the persistent presence of the residual deletion mutant RLIP76. Western blotting against the anti-del 171-185 antibody showed no signal in the RLIP76 deletion mutant transfected cells confirming that expression of wild-type RLIP76 was effectively blocked in these cells. Cell surface expression of RLIP76 in del 171-185 transfected cells with or without pre-treatment with si-RNA directed at aa 171-185 using immunohistochemical analaysis and an anti-del 171-185 antibody showed that cells with control si-RNA had significant cell surface signal which was absent in cells in which RLIP had ben silenced by the si-RNA. RLIP76 is, thus, an integral membrane protein with at least one cell surface domain spanning amino acids 154 to 219.

Accordingly, the present invention provides several surface epitope regions of RLIP that, when altered, blocked or deleted, prevent RLIP from performing its transport function. Compositions of the present invention include the several surface epitope regions as well as use of these surface epitope regions to obtain specific inhibitors of RLIP that are capable of altering, inhibiting or the transport function of RLIP. The inhibitors include si-RNAs, each having a sequence directed against the one or more surface epitope regions as well as phosphorothioate antisense oligonucleotides directed against such surface epitope regions (e.g., GGCTCCTGAATTGGCTTTTTC; SEQ ID NO.:18) and a corresponding silencing RNA sequence to the phosphorothioate antisense oligonucleotides (e.g., AAGAAAAGCCAATTCAGGAGCC; SEQ ID NO.:19). In addition, inhibitors include antibodies (monoclonal and/or polyclonal) directed against the one or more surface epitope regions, such regions including SEQ ID NO.: 3 and SEQ ID NO.:4. Moreover, the inhibitors identified herein provide compounds for anti-seizure medicines. Importantly, the inhibitors are additional targets for identifying important compounds and small molecule from chemical library screenings, wherein the identified compounds and/or small molecules are effective as anti-seizure medicines.

While particular embodiments of the invention and method steps of the invention have been described herein, additional alternatives not specifically disclosed but known in the art are intended to fall within the scope of the invention. Thus, it is understood that other embodiments and applications of the present invention will be apparent to those skilled in the art without departing from the scope and spirit of the appended claims and drawings.

What is claimed is:

1. A composition comprising:
a coding region of ralA binding protein 1, wherein the region further comprises SEQ ID NO.:20 and modified variants thereof, including deletions of 1 to 15 nucleotides at its C-terminus and N-terminus.

2. The composition of claim 1, wherein the composition is used to identify antibodies that effect transport activity and membrane association of ralA binding protein 1.

3. The composition of claim 1, wherein the composition is used to identify compounds that recognize ralA binding protein 1.

4. The composition of claim 3, wherein the compounds directly effect transport activity and membrane association of ralA binding protein 1.

5. The composition of claim 3, wherein the compounds are anti-seizure medicines.

6. The composition of claim 1, wherein the composition codes for SEQ ID NO.:3 and modified variants, thereof.

7. The composition of claim 6, wherein SEQ ID NO.:3 and modified variants thereof are used to generate antibodies to ralA binding protein 1.

8. The composition of claim 6, wherein SEQ ID NO.:3 and modified variants thereof are used for screening chemical libraries for compounds that recognize SEQ ID NO.:3.

9. The composition of claim 8, wherein the compounds are anti-seizure medicines.

10. The composition of claim 1, wherein the composition codes for SEQ ID NO.:4 and modified variants.

11. The composition of claim 10, wherein SEQ ID NO.:4 and modified variants thereof are used to generate antibodies to the ralA binding protein 1.

12. The composition of claim 10, wherein SEQ ID NO. :4 and modified variants thereof are used for screening chemical libraries for compounds that recognize SEQ ID NO.:4.

13. The composition of claim 1, wherein the composition further comprises SEQ ID NO.:21 and modified variants thereof, including deletions of 1 to 15 nucleotides at its C-terminus and N-terminus.

14. The composition of claim 13, wherein SEQ ID NO.:21 is used to identify antibodies that effect transport activity and membrane association of ralA binding protein 1.

15. The composition of claim 14, wherein SEQ ID NO.:21 is used to identify compounds that recognize the ralA binding protein 1.

16. The composition of claim 15, wherein the compounds are anti-seizure medicines.

17. The composition of claim 1, wherein the composition is used to create phosphorothioate antisense oligonucleotides directed to the ralA binding protein 1.

18. The composition of claim 1, wherein the composition is used to create si-RNA sequences to silence expression of wild-type ralA binding protein 1.

19. A method comprising the steps of:
providing to a subject in need thereof a derived composition provided by the composition of claim 1, wherein the derived composition is an antibody, si-RNA, small molecule and combinations thereof that directly effects transport activity and membrane association of a ralA binding protein 1 at the blood-brain barrier.

20. The composition of claim 19, wherein the derived composition is used alone or in combination with anti-seizure medicines.

21. The composition of claim 19, wherein the derived composition is identified by screening chemical libraries for compounds that recognize the ralA binding protein 1.

22. The composition of claim 19, wherein the derived composition is obtained by using it to raising antibodies to ralA binding protein 1.

23. A composition comprising:
a coding region of ralA binding protein 1, wherein the region further comprises SEQ ID NO.:3 and modified variants thereof.

24. The composition of claim 23, wherein the composition is used to identify antibodies that effect transport activity and membrane association of ralA binding protein 1.

25. The composition of claim 23, wherein the composition is used to identify compounds that recognize SEQ ID NO.:3.

26. The composition of claim 23, wherein the compounds directly effect transport activity and membrane association of ralA binding protein 1.

27. The composition of claim 25, wherein the compounds are anti-seizure medicines.

28. The composition of claim 23, wherein the composition is used to generate antibodies to SEQ ID NO.:3.

29. The composition of claim 23, wherein the composition is used for screening chemical libraries for compounds that bind SEQ ID NO. :3.

30. The composition of claim 23 further comprising SEQ ID NO.:4.

31. The composition of claim 23, wherein the composition is used to identify phosphorothioate antisense oligonucleotides directed to the nucleotide portion of SEQ ID NO.:3.

32. The composition of claim 23, wherein the composition is used to identify si-RNA sequences that silence expression of wild-type ralA binding protein 1.
